(19) United States
(12) Patent Application Publication
(10) Pub. No.: US 2015/0038891 A1
(43) Pub. Date: Feb. 5, 2015

(54) PROTECTIVE SHEATH

(71) Applicants: James Lipton, New York, NY (US); Ivan R. Sabel, Washington, DC (US)

(72) Inventors: James Lipton, New York, NY (US); Ivan R. Sabel, Washington, DC (US)

(21) Appl. No.: 14/448,503

(22) Filed: Jul. 31, 2014

Related U.S. Application Data
(60) Provisional application No. 61/861,007, filed on Aug. 1, 2013.

(51) Int. Cl. A61F 5/01

(52) U.S. Cl. CPC A61F 5/0109
USPC 602/26

(57) ABSTRACT

This invention relates generally to providing relief to persons who suffer from arthritis of the knee and/or other knee ailments, and in particular to a soft, flexible sheath for protecting the knee and providing comfort to the user while sleeping. Various embodiments are described, which are constructed so that the wearer can bend and straighten his/her knees.

### RELATED APPLICATION

This application claims the benefit of, and priority to, U.S. Provisional Application No. 61/861,007, filed Aug. 1, 2013, entitled “Protective Sheath,” which is incorporated herein by reference in its entirety.

### BACKGROUND

Millions of men and women suffer arthritis of the knee(s). Arthritis of the knees can be the result of injury, misuse, or simply the passage of time, but once it begins, so does the search for relief, which takes many forms, including medication, exercise, surgery, and rehabilitation. With arthritis, or injury to the knee(s), even sleeping is a challenge: often, the person wakes over and over again, because of a stabbing pain in one or both knees.

The relief suggested by medical care professionals is to place a pillow between the knees. However, the use of a pillow is less than satisfactory. For example, when the person falls asleep, the pillow typically becomes dislodged and the stabbing pain returns, particularly as the knees come into contact with each other. Accordingly, an alternative solution is desirable.

### SUMMARY

The present invention provides, in various embodiments, a soft, flexible sheath constructed to be worn by a person with arthritis of the knee and/or other knee ailments while sleeping. The protective sheath of the present invention can slide up onto the person's leg, to cover the affected knee yet allow the knee to be articulated, and can remain in position throughout the night whether the person sleeps on his/her side, back, or stomach.

In one aspect, the invention provides a protective sheath configured to surround and extend above and below a user's knee and to provide cushioning of at least 0.5″ on either side of the knee to protect the knee when the user is sleeping.

In some embodiments, the sheath comprises a front portion configured to be positioned at the front of the knee and a back portion configured to be positioned behind the knee, the front portion and the back portion securely attached together to surround the user's knee closely.

In some embodiments, the front portion comprises a main body. In some embodiments, the main body comprises an outer face and an inner face, the inner face having two cushioned sections attached thereto and configured to be positioned at each side of the user's knee. In some embodiments, the main body comprises two or more panels sewn together. In some embodiments the main body includes an open portion configured to be positioned over the user's kneecap.

In various embodiments, the front portion includes one or more cutouts at each side of the main body; side flaps securely attached at each side of the main body; and/or end flaps securely attached at each end of the main body.

In some embodiments, the front portion is made from synthetic rubber. In some embodiments, the back portion is made from elastic nylon.

In some embodiments, the front portion is configured to form an arch lengthwise and widthwise, providing a double curvature that conforms to a bent or relaxed knee.

In some embodiments, the sheath comprises a rectangular panel made of foam rubber or sponge. In some embodiments, the panel has a thickness of about 0.5″ to 1.5″. In some embodiments, the panel has two short ends securely attached together to form a cylindrical sheath. In some embodiments, the cylindrical sheath has an inner diameter that is at least 0.5″ to 2.0″ greater than a diameter of the user's knee.

Additional features and advantages of the present invention are described further below. This summary section is meant merely to illustrate certain features of the invention, and is not meant to limit the scope of the invention in any way. The failure to discuss a specific feature or embodiment of the invention, or the inclusion of one or more features in this summary section, should not be construed to limit the invention as claimed.

### BRIEF DESCRIPTION OF THE DRAWINGS

The foregoing summary, as well as the following detailed description of the preferred embodiments of the application, will be better understood when read in conjunction with the appended drawings. For the purposes of illustrating the device of the present application, there are shown in the drawings exemplary embodiments. It should be understood, however, that the application is not limited to the precise arrangements and instrumentalities shown. In the drawings:

FIG. 1A is an elevational view of a front section of a protective sheath in accordance with an exemplary embodiment of the invention;

FIG. 1B is an elevational view of the back of the protective sheath of FIG. 1A;

FIG. 2 is a side view of the protective sheath of FIG. 1A;

FIG. 3 is an end view of the protective sheath of FIG. 1A;

FIG. 4 is an elevational view of the inside face of the front section shown in FIG. 1A;

FIG. 5 is a side view of the protective sheath of FIG. 1A worn by a person with his/her leg bent.

FIG. 6 is a plain view of a protective sheath positioned over the knee of a person in accordance with another exemplary embodiment of the invention;

FIG. 7 is an elevational view of the sheath of FIG. 6 before it is assembled;

FIG. 8 is an elevational view of the protective sheath assembled from the material illustrated in FIG. 7.

FIG. 9 is a sectional view of the protective sheath taken along line 9-9 of FIG. 8;

FIG. 10 is a sectional view of the protective sheath taken along line 10-10 of FIG. 6 surrounding a person's knee when the person is lying on his/her back.

### DETAILED DESCRIPTION

The present invention provides, in various embodiments, a soft protective sheath that is adapted to surround the knee and protect the lateral and medial condyles, and permit the bending and straightening of the knee when the person is in bed. The protective sheath will substantially reduce contact between the knees and will reduce the piercing pain that occurs in the knee preventing the person from sleeping. In some embodiments, the protective sheath is adapted to be slid over a person's knee or knees and provide protection without vascular constriction. The protective sheath is configured to remain in place when the person is in bed, but is easily removable when the person is not in bed.

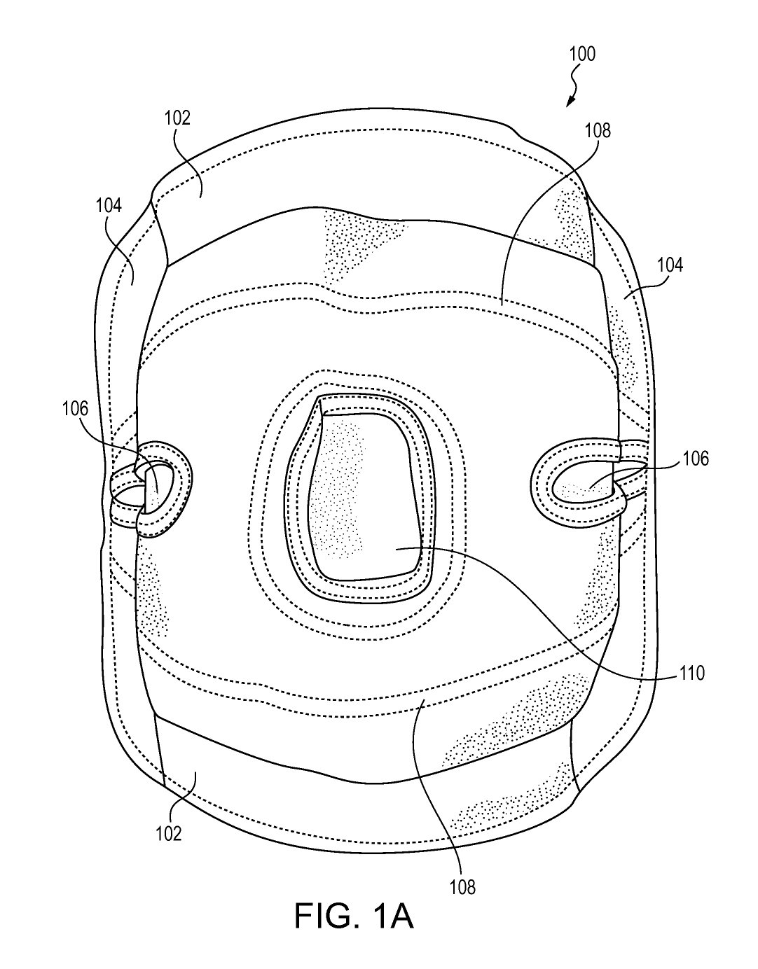

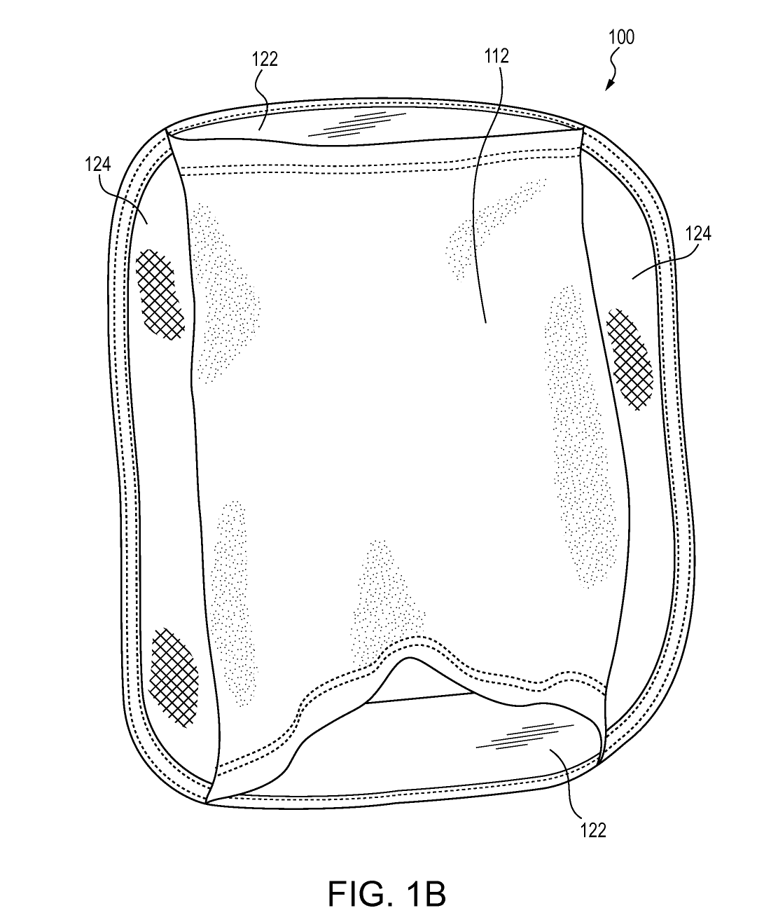

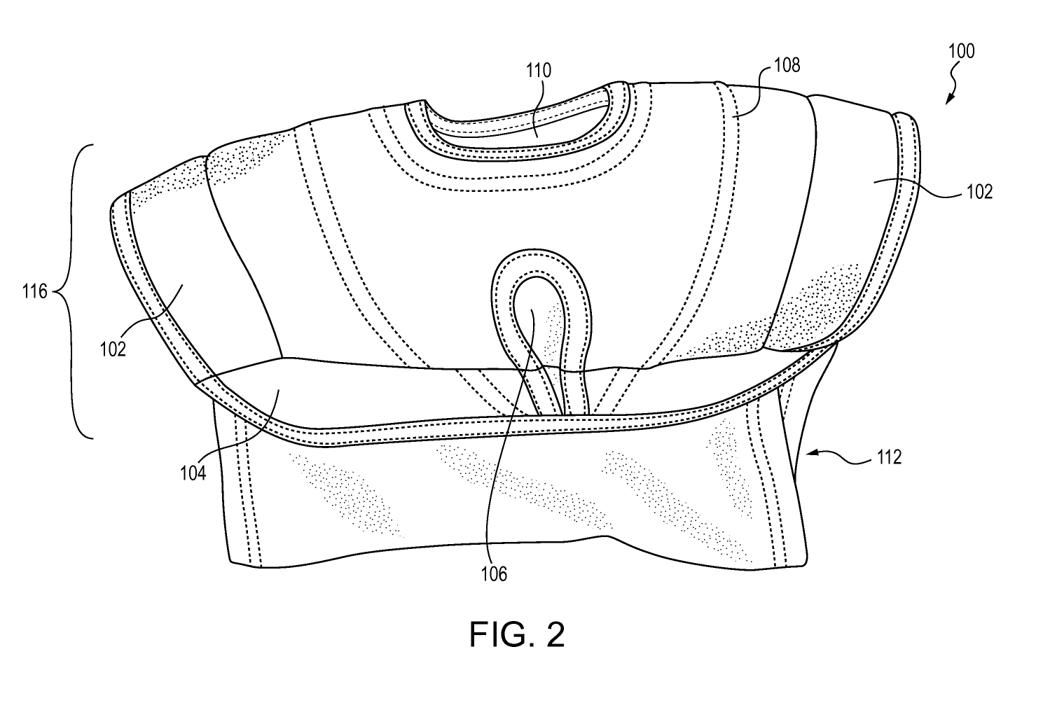

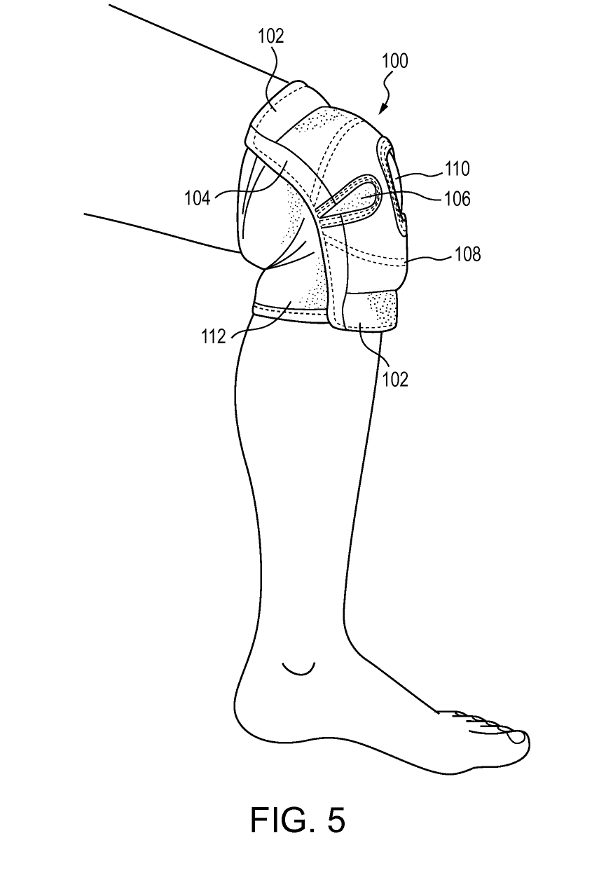

One illustrated embodiment of the present invention relates to a fitted flexible sheath to be worn around a person's knee. Advantageously, a variety of different materials are combined to protect the medial and lateral aspects of the knee, while still allowing full range of motion and slippage control without restricting circulation. Reference is made to FIGS. 1A-B, 2, 3, 4 and 5, which depict an exemplary embodiment of a protective sheath, generally indicated as 100. As depicted in the side views of FIG. 2 and FIG. 5, sheath 100 has a front portion 116 and a back portion 112. As shown in FIG. 5, when sheath 100 is in use, the front shell covers the knee, and the back panel fits snugly around the back of the leg. An open area 110 may be provided in front portion 116, which can be positioned over the kneecap to allow the knee to bend comfortably and minimize pressure on the kneecap while wearing sheath 100. Area 110 may vary in shape and/or size. In some embodiments, open area 110 is substantially oval, having a length through the center of the opening of about 1.0″ to 4.0″, preferably about 2.25″, and a width through the center of the opening of about 1.0″ to 3.0″, preferably about 1.75″.

The front portion of sheath 200 is preferably made of a soft, sturdy, flexible material, such as, but not limited to, neoprene. As shown in FIGS. 1A, 2, and 5, in some embodiments, front portion 116 comprises several panels sewn together or securely attached by other means. In some embodiments, front portion 116 includes a main body and side flaps 104 and/or end flaps 102. Side cutouts 106 on the main body may also be provided. The main body may comprise two or more panels joined together by stitching or other secure attachment means at one or more seams 108. Side flaps 104, end flaps 102, cutouts 106, seams 108, and opening 110 function individually, together, or in any sub-combination to allow the leg to rest comfortably, and bend and straighten easily, when sheath 100 is worn.

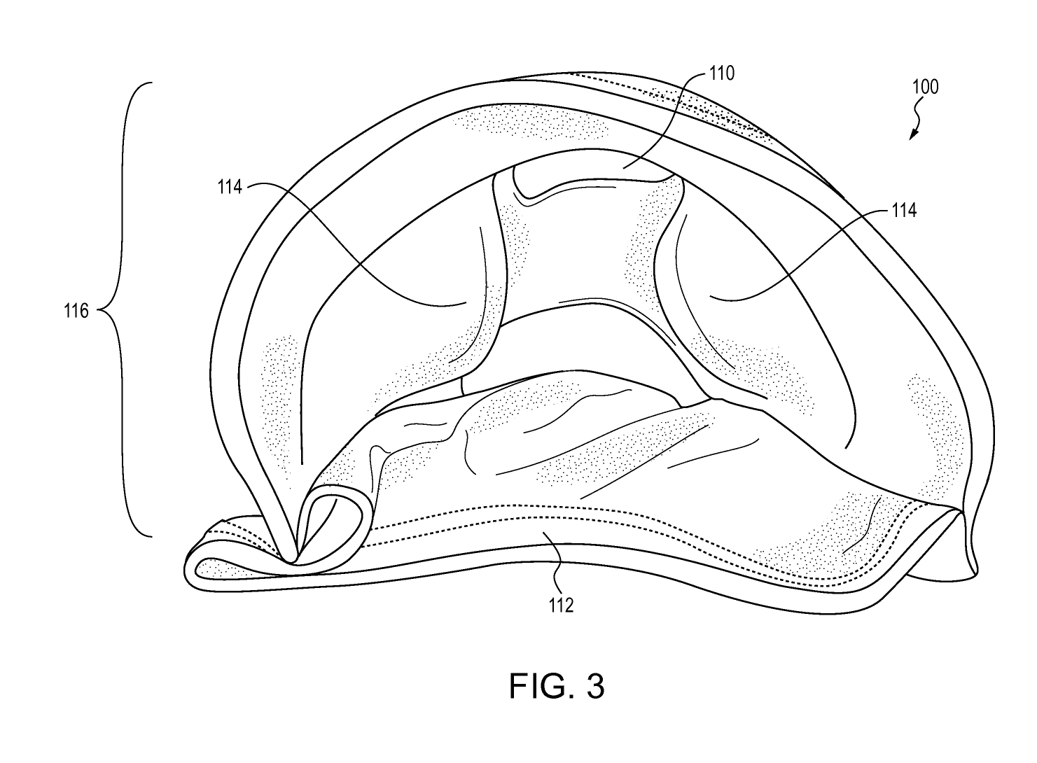

Sheath 100 is constructed to fit closely and extend around the knee, for example as shown in FIG. 5. In some embodiments, due to the cut of the panels and/or placement of the seams, front portion 116 has a curvature or arch along its length, which complements a bent or relaxed knee. For example, as depicted in FIG. 2, the length across the top of front portion 116 is greater than the length along the bottom. In an exemplary embodiment, the main body of front portion 116 (excluding end flaps 102) is approximately 7.5″ long down the center, and approximately 6.0″ at each side. Front portion 116 is also preferably curved along its width, as shown in FIG. 3, to cradle the knee from side to side. In an exemplary embodiment, the main body of front portion 116 (excluding side flaps 104) is approximately 7.5″ wide across the center, and approximately 6.5″ wide at each end.

As shown in FIG. 1B, in some embodiments, back portion 112 is sewn to front portion 116 along the inner edges of side flaps 104. Back portion 112 is preferably made of a thin, flexible material, such as, but not limited to, an elastic nylon (e.g., a nylon/spandex blend), and helps hold front portion 116 in place over the knee. Unlike other mechanisms for positioning front portion 116 (e.g., adjustable straps), back portion 112 is of a size and flexibility such that sheath 100, while configured to be snugly fitted, does not restrict blood flow. Advantageously, different sizes of sheath 100 (e.g., small, medium, large) can be produced by using different sizes of back portion 112 attached to one size of front portion 116. In some embodiments, the same or similar material is used to cover the area behind cutouts 106. Side panels 104 and/or end panels 102 may be covered/reinforced on the back side with another material (different from neoprene or other material used for front portion 116). In some embodiments, the backs of side panels 104 are lined with a cushioned fabric 124 (e.g., a flexible cloth with a thin layer of foam backing, such as an open cell foam about ⅛″ thick), and the backs of end panels 102 are reinforced with an elastic band 122.

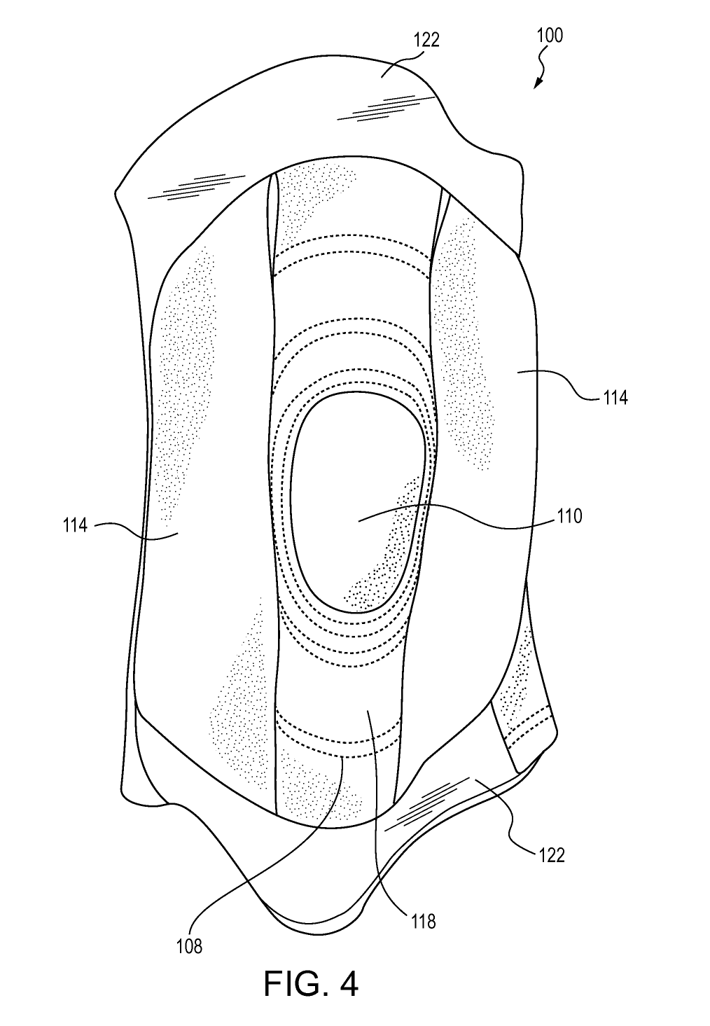

As shown in the end view of FIG. 3, side cushions 114 are provided on the sides of sheath 100 to protect both sides of the knee. Side cushions 114 are made of foam rubber or sponge material, such as, but not limited to, memory foam or a lower cost equivalent, optionally rounded and shaped to fit the inner face of front portion 116 as shown in FIG. 4. In some embodiments, side cushions 114 comprise memory foam, which can be shaped better, will conform better, and/or will more likely retain its shape as compared to other foam materials. Side cushions 114 have a thickness sufficient to cushion the knee, for example approximately 0.5″ to 2.5″ and in an exemplary embodiment about 1.5″. Side cushions 114 need not have a uniform thickness, and in some embodiments may be shaped so that they are thicker in the center than at the edges.

In some embodiments, the face of side cushions 114 that will contact the person's knee is covered with a cushioned fabric (e.g., like the back sides 124 of side flaps 104). As depicted in the inside-out view of FIG. 4, in some embodiments, a central lengthwise segment of front shell 116 is unlined neoprene 118, and the backs of end panels 102 are reinforced with an elastic band 122 (as also shown in FIG. 1B). In some embodiments, a non-slip rubber inset may be included at the top and bottom of the front shell, which will ensure that it stays in place regardless of movement. Side cushions 114 extend along the underside of front shell 116, bordered by the inner seams of end panels 102 and side panels 104 and preferably not extending beyond open portion 110 in the center.

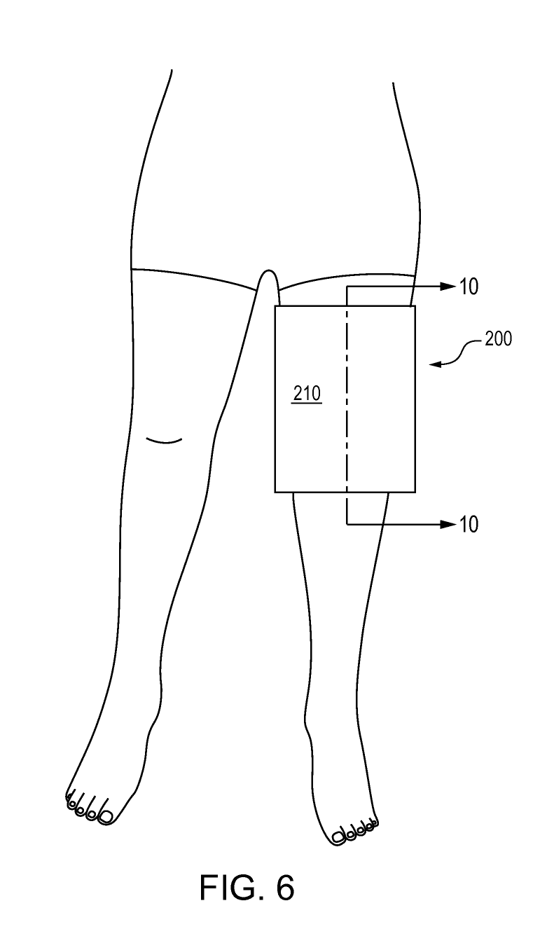

Another illustrated embodiment of the present invention relates to a soft cylindrical sheath to be worn around a person's knee. Reference is made to FIGS. 6, 8, 9 and 10, which depict an exemplary embodiment of a protective sheath, generally indicated as 200. As depicted in FIG. 8, sheath 200 includes a first opening 202 and a second opening 204 so that the sheath can slide over a person's knee and rest in a position extending slightly below the knee and slightly above the knee to provide protection to the knee when a person is trying to rest or sleep.

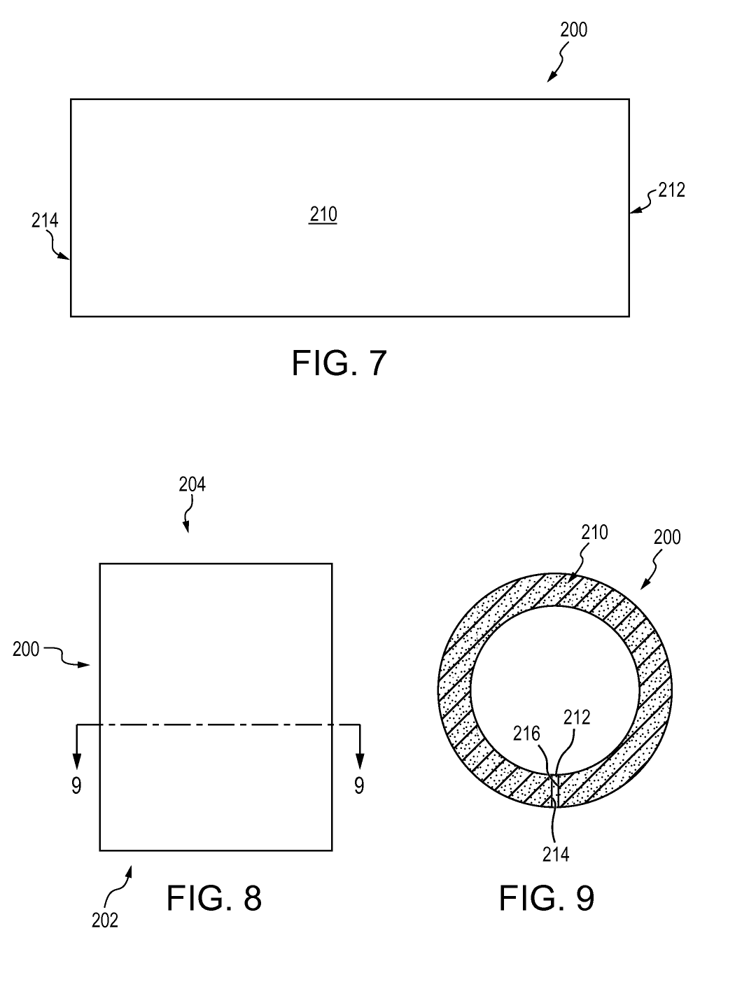

One embodiment of sheath 200 is depicted in FIG. 7, wherein the sheath is made from a substantially rectangular panel of foam rubber or sponge material. The foam rubber or sponge material should have a thickness sufficient to cushion the knee, for example approximately 0.5″ to 1.5″ and in an exemplary embodiment preferably 1.0″. As depicted in FIGS. 7 and 9, panel 210 is provided with a first end 212, and a second end 214, that are joined together by a suitable mechanical fastening mechanism 216 such as hot melt glue to form a cylindrical sheath. Other mechanical mechanisms such as stitching can be used to secure ends 212 and 214 together to form sheath 200. In alternative embodiments, hook and loop fasteners or snap fasteners can be used on first end 212 and second end 214 of panel 210 to permit the ends to be releasably secured together to permit sheath 200 to be formed by wrapping panel 210 around the knee and securing both ends together to form the protective sheath and thereby avoid having to slide a pre-formed sheath over the knee. In still a further embodiment, sheath 200 can be extruded in order to provide a one-piece tubular configuration in accordance with the invention.

As shown in FIG. 6, tubular sheath 200 covers the knee and offers it cushioning when the person is on his/her back. Advantageously, by having the inside diameter of protective sheath 200 greater than the outside diameter of the knee, at any portion of the knee that is covered by the sheath, the knee is given room to be bent or straightened and hence the protective sheath will remain on the knee during sleep, yet can be easily removed by the user.

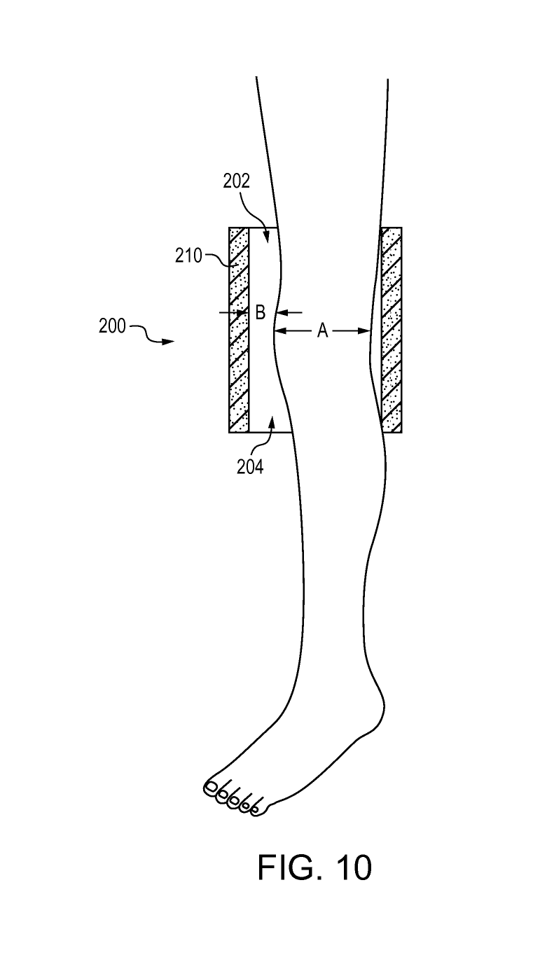

The inside diameter of sheath 200 is preferably at least 0.5″ to 2.0″ greater than the diameter of the knee. In an exemplary embodiment it should be approximately 1.0″ greater in diameter than the person's knee to ensure that there is sufficient room for the knee to be bent and articulated by the person during rest and sleep. As depicted in FIG. 10, in an exemplary embodiment sheath 200 allows a space B beyond the diameter of the knee A.

While there have been shown and described fundamental novel features of the invention as applied to the preferred and exemplary embodiments thereof, it will be understood that omissions and substitutions and changes in the form and details of the disclosed invention may be made by those skilled in the art without departing from the spirit of the invention. Moreover, as is readily apparent, numerous modifications and changes may readily occur to those skilled in the art. Hence, it is not desired to limit the invention to the exact construction and operation shown and described and, accordingly, all suitable modification equivalents may be resorted to falling within the scope of the invention as claimed. It is the intention, therefore, to be limited only as indicated by the scope of the claims appended hereto.

What is claimed is:

1. A protective sheath configured to surround and extend above and below a user's knee and to provide cushioning of at least 0.5″ on either side of the knee to protect the knee when the user is sleeping.

2. The protective sheath of claim 1, wherein the sheath comprises a front portion configured to be positioned at the front of the knee and a back portion configured to be positioned behind the knee, the front portion and the back portion securely attached together to surround the user's knee closely.

3. The protective sheath of claim 2, wherein the front portion comprises a main body.

4. The protective sheath of claim 3, wherein the main body comprises an outer face and an inner face, the inner face having two cushioned sections attached thereto and configured to be positioned at each side of the user's knee.

5. The protective sheath of claim 3, wherein the main body comprises two or more panels sewn together.

6. The protective sheath of claim 3, wherein the main body includes an open portion configured to be positioned over the user's kneecap.

7. The protective sheath of claim 3, wherein the front portion includes one or more cutouts at each side of the main body.

8. The protective sheath of claim 3, wherein the front portion includes side flaps securely attached at each side of the main body.

9. The protective sheath of claim 3, wherein the front portion includes end flaps securely attached at each end of the main body.

10. The protective sheath of claim 2, wherein the front portion is made from synthetic rubber.

11. The protective sheath of claim 2, wherein the back portion is made from elastic nylon.

12. The protective sheath of claim 2, wherein the front portion is configured to form an arch lengthwise and widthwise, providing a double curvature that conforms to a bent or relaxed knee.

13. The protective sheath of claim 1, wherein the sheath comprises a rectangular panel made of foam rubber or sponge.

14. The protective sheath of claim 13, wherein the panel has a thickness of about 0.5″ to 1.5″.

15. The protective sheath of claim 13, wherein the panel has two short ends securely attached together to form a cylindrical sheath.

16. The protective sheath of claim 13, wherein the cylindrical sheath has an inner diameter that is at least 0.5″ to 2.0″ greater than a diameter of the user's knee.
